(19) United States
(12) Patent Application Publication
(10) Pub. No.: US 2025/0090714 A1
(43) Pub. Date: Mar. 20, 2025

(54) PLASMA AIR PURIFYING APPARATUS

(71) Applicant: JUNSYS Co., Ltd., Gunpo-si (KR)

(72) Inventors: Deoksoo JUN, Gwangmyeong-si (KR); Yanghyun CHOI, Seoul (KR)

(21) Appl. No.: 18/795,800

(22) Filed: Aug. 6, 2024

(30) Foreign Application Priority Data
Sep. 18, 2023 (KR) 10-2023-0123624

(51) Int. Cl. A61L 9/20

(52) U.S. Cl. CPC A61L 9/20; A61L 2209/111; A61L 2209/14

(57) ABSTRACT

The present invention relates to an apparatus for purifying indoor air using plasma. More specifically, the plasma air purifying apparatus according to an embodiment of the present invention includes a plasma discharge module that generates plasma to remove microorganisms such as bacteria and viruses and various harmful substances contained in the sucked air, a catalytic filter unit that removes residual ozone (O₃) remaining after a chemical reaction by plasma, an ultraviolet (UV) irradiation unit that rapidly promotes the ozone (O₃) decomposition reaction of the catalytic filter unit, an ozone sensor unit that detects the concentration of ozone (O₃) generated by plasma, a power supply unit that supplies electric power to the plasma discharge module, and a control unit that controls the power supply of the power supply unit.

### TECHNICAL FIELD

The present invention relates to a plasma air purifying apparatus. Particularly, the present invention relates to a plasma air purifying apparatus comprising an ozone (O₃) decomposition catalyst.

More specifically, the present invention relates to a method for manufacturing an apparatus that generates a low temperature plasma in an atmospheric pressure state to simultaneously removes microorganisms and harmful substances contained in the air. More specifically, the present invention relates to a method for manufacturing an atmospheric pressure plasma air purifying apparatus in which the high sterilizing power of active species in an ionized state such as a hydroxyl radical (OH radical) and an active oxygen generated by plasma discharge in an atmospheric pressure state is used to kill bacteria and viruses, and remove substances harmful to the human body, such as ammonia, hydrogen sulfide, formaldehyde, and the like, thereby providing a structure and a control method which prevent the deterioration of plasma performance caused by side effects which are generated by continuously applying strong energy to the plasma discharge electrode due to continuous operation.

In addition, the present invention relates to a method that facilitates the production of a catalytic filter and the catalytic reaction required to rapidly decompose inside a plasma air purifying apparatus, decrease its concentration to an environmental standard value or lower, and discharge it as purified air because the ozone (O₃) generated during plasma discharge in atmospheric air may be harmful to a human body.

### BACKGROUND ART

Air contaminated with harmful substances such as various pathogenic bacteria including COVID-19, viruses, volatile organic compounds (VOCs), bad smells, and fine dust is recognized as a serious social problem that threatens people's daily lives. In order to improve such problems, usage of the air purifying apparatus products that help improve the air quality in our living environment is on an increasing trend.

The dust collection filter type air cleaning machine commonly used in the art are used for the purpose such as collection of floating powder dust contained in the air in order to maintain the cleaning state of indoor air, but due to the limitations of the built-in filter performance, the effect of removing pathogens such as various bacteria, viruses, and mold is limited.

In contrast, in the case of an air purifying apparatus that uses plasma, when high energy is applied to oxygen, water vapor, and other molecules present in the air, they become in a plasma state where they are separated into a positive ion and a negative ion. The hydroxyl radical (OH radical) and active oxygen generated in this process combine with hydrogen ions (H⁺), which are the main components of bacteria and viruses, to destroy cell membranes, thereby providing an environmentally friendly sterilizing effect which kills them and reduces them back to water (H₂O) and oxygen (O₂).

It is known that the apparatus is effective in decomposing various harmful substances in addition to such pathogenic microorganism removing functions and thus, can be beneficial to human life by removing pollutants such as volatile organic compounds (VOCs), bad smells, and fine dust.’

In order to obtain excellent effects by applying plasma to an air purifying apparatus in the air, plasma must be generated with an output appropriate for the environment in which the apparatus is used. At this time, as strong energy is continuously applied to a plasma discharge electrode, the discharge electrode disappears to cause a performance degradation problem, and a side effect occurs in that ozone (O₃), which is a gas harmful to the human body, is generated.

Due to such problems, the plasma discharge output has no choice but to be weakly limited, which makes it difficult to obtain a good air purifying effect, and some products use activated carbon adsorbents as a method for removing ozone (O₃) generated at this time, but it is confirmed that the ozone (O₃) removal effect is limited.

Recently, various plasma sterilization and deodorization apparatuses have been disclosed, such as Korean Patent No. 10-2427415 entitled “Sterilization and deodorization system using ozone and low temperature plasma photocatalyst” which has been presented as a way to solve these problems. However, although efforts have been made to increase the sterilization and deodorization effect by using ultraviolet (UV) light and photocatalysts in addition to plasma, a method for effectively removing ozone (O₃), a harmful gas generated in the inside by plasma discharge, has not been clearly presented.

### DETAILED DESCRIPTION OF THE INVENTION

### Technical Problem

The present invention has been designed to solve the above-mentioned problems, and therefore, an object of the present invention is to provide an air purifying apparatus having improved life and air purifying performance.

Another object of the present invention is to provide a method for manufacturing an air purifying apparatus at a lower cost.

### Technical Solution

In order to achieve the above object, an air purifying apparatus according to the present invention comprises a plasma reaction unit equipped with at least two or more plasma discharge modules that generate plasma to purify air contaminated with harmful substances such as various pathogenic bacteria, viruses, bad smells and fine dust contained in the air, a catalytic filter unit that communicates therewith to remove ozone (O₃) inside, an ultraviolet (UV) irradiation unit that rapidly promotes the ozone (O₃) decomposition reaction of the catalytic filter unit, a power supply unit that supplies electric power to the plasma discharge module, and a control unit that controls the electric power supply of the power supply unit, wherein the control unit is controlled so that at least two or more plasma discharge module receive supply of electric power at a fixed cycle and in sequence, but provide continuity as a whole.

Wherein the plasma discharge module is generally composed of two or more even numbers, and can be symmetrically installed in the front-rear and left-right directions with respect to the center of the plasma reaction unit.

On the other hand, a catalytic agent of the catalytic filter unit, which has the function of removing residual ozone (O₃) remaining after the chemical reaction in the plasma reaction unit, is a pellet or ball-shaped catalyst having a diameter of 5 mm or less containing 72 to 84 mol % of manganese dioxide (MnO₂) and 16 to 28 mol % of copper oxide (CuO), and includes a flat honeycomb filter having a structure that allows gas to pass therethrough and in which the compound catalytic agent is incorporate, the compound catalytic agent gas-passing catalytic filter in which a manganese dioxide (MnO₂)-copper oxide (CuO) compound catalytic agent is incorporated, the compound catalytic agent having a structure wherein titanium dioxide (TiO₂) particles having a diameter of 100 nm or less are evenly applied or coated on the surface of the catalytic agent at 50 to 80%.

In addition, in order to more quickly promote the ozone (O₃) decomposition reaction of the catalytic filter unit, ultraviolet (UV) is irradiated to the catalytic agent, wherein the ultraviolet (UV) light generation device irradiates an ultraviolet (UV) by using at least four semiconductor LEDs with a light source wavelength of 250 nm to 400 nm and an ultraviolet (UV) radiation output of 10 mW to 1,000 mW, thereby increasing the activity of the catalytic reaction and exhibiting the function of promoting the ozone (O₃) decomposition reaction.

Wherein an ozone sensor is provided to detect the concentration of residual ozone (O₃) remaining in the inside air after the chemical reaction in the plasma reaction unit, and based on the detected concentration of ozone (O₃), the operating time of the plasma discharge module is adjusted, and the irradiation intensity and quantity of ultraviolet (UV) light that enhances the activity of the catalytic reaction are automatically adjusted.

### Advantageous Effects

According to the present invention, it is possible to improve the structure and control method of the plasma discharge module, thereby extending its life and increasing its performance.

In addition, titanium dioxide (TiO₂) particles are coated onto the surface of the manganese dioxide (MnO₂) compound catalytic agent built into the catalytic filter unit and irradiated with ultraviolet (UV) light, thereby increasing the activity of the catalytic reaction and rapidly removing ozone (O₃) from the inside.

As a result, it is possible to significantly reduce the apparatus production cost compared to performance.

Effects of the present invention are not limited to the effects mentioned above, and additional other effects not mentioned herein will be clearly understood from the detailed description and the appended claims by those skilled in the art.

### BRIEF DESCRIPTION OF THE DRAWINGS

FIG. 1 is a perspective view of a plasma air purifying apparatus according to one embodiment;

FIG. 2 is a block diagram schematically showing the internal components of FIG. 1;

FIG. 3 is a block diagram showing the cross-sectional structure of the arrangement of the plasma discharge module shown in FIG. 2;

FIG. 4 is an exemplary diagram showing the plasma discharge module shown in FIG. 3;

FIG. 5 is an exemplary diagram showing a method of applying electric power to the plasma discharge module shown in FIG. 3;

FIG. 6 is a block diagram of the catalytic filter unit and ultraviolet (UV) irradiation unit shown in FIG. 2;

FIG. 7 is a graph showing the ozone decomposition rate of the catalytic filter unit shown in FIG. 6; and

FIG. 8 is a test result statement data showing the results of a sterilization test by a plasma air purifying apparatus according to one embodiment.

### DETAILED DESCRIPTION OF THE EMBODIMENTS

Now, a plasma air purifying apparatus according to a preferred embodiment will be described with reference to the accompanying drawings.

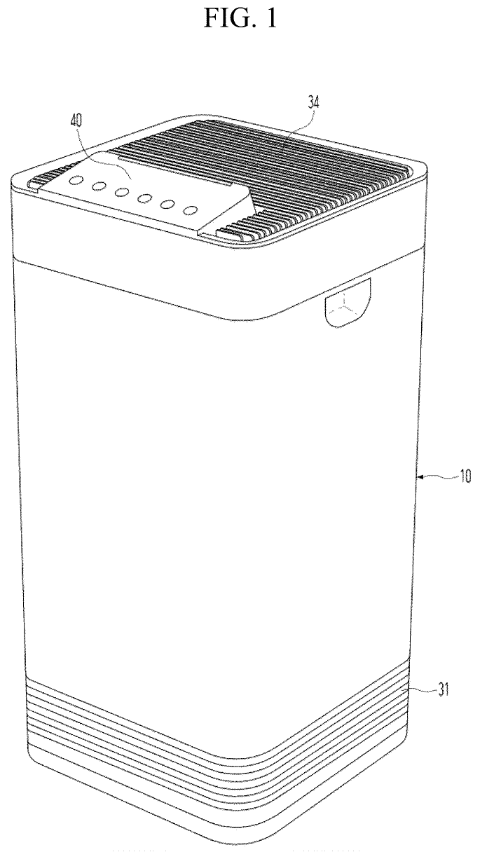

FIG. 1 is a perspective view of a plasma air purifying apparatus according to one embodiment.

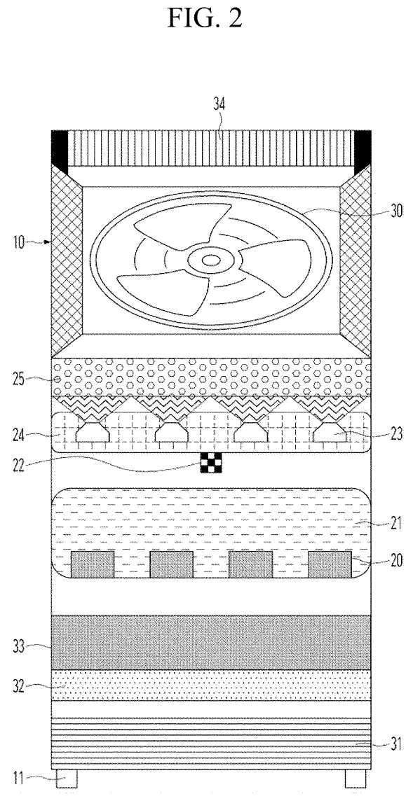

Referring to FIGS. 1 and 2, a plasma air purifying apparatus according to one embodiment may include a housing 10 that includes the main components such as a plasma reaction unit 21, a catalytic filter unit 25, an ultraviolet irradiation unit 24, a blower fan 30, a pre-filter 32, a HEPA filter 33, and a device operating unit 40 inside, wherein a stand 11 may be installed on the four lower corners of the housing, or a caster may be formed as a wheel that can be moved by pushing or pulling the device.

Here, the shape of the housing 10 can be constituted in various shapes, but it is preferable to be constituted in a square or cylindrical shape formed upright, but it is not limited thereto, and it can be of course produced in various shapes such as a sphere or pyramid shape.

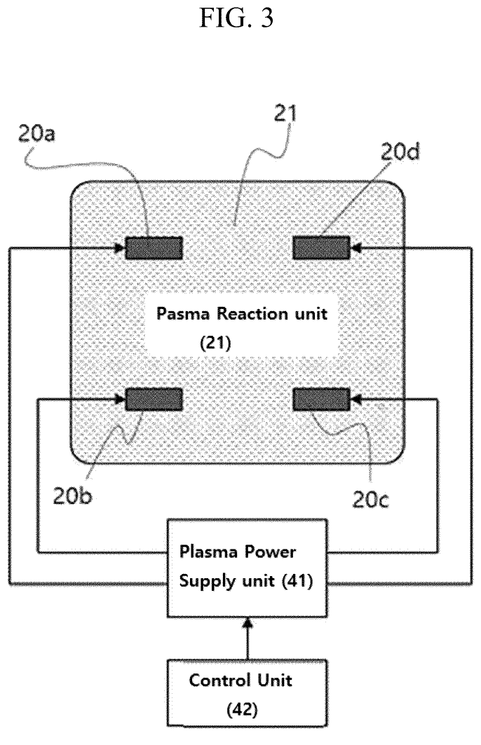

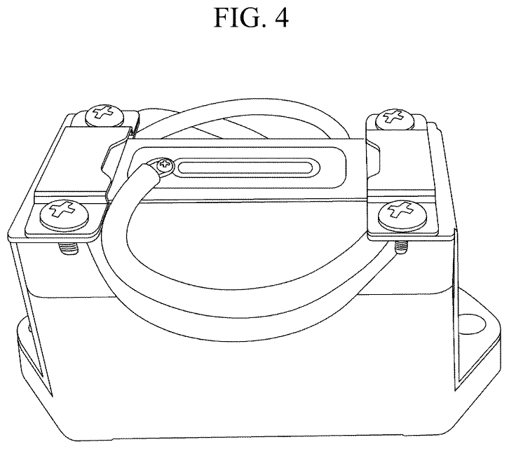

FIG. 2 is a block diagram schematically showing the internal components of FIG. 1, FIG. 3 is a block diagram showing the cross-sectional structure of the arrangement of the plasma discharge modules 20a˜20d shown in FIG. 2, and FIG. 4 is an exemplary diagram showing one embodiment of the plasma discharge module shown in FIG. 3.

The plasma discharge modules 20a˜20d are devices for generating plasma in an atmospheric pressure state within the plasma reaction unit 21, wherein a plurality of plasma discharge modules 20a˜20d are arranged symmetrically with respect to the internal center of the plasma reaction unit 21, and preferably consist of four modules, which can be arranged symmetrically in the front-rear and left-right directions of the plasma reaction unit 21. In this case, the chemical reaction by plasma can be evenly diffused and acted on the air to be purified passing through the plasma reaction unit 21, which is expected to provide good performance and increase the electric power supply efficiency of the plasma power supply unit 41 described below.

On the other hand, if the capacity of the air purifying apparatus is large and a larger generating amount of plasma is required, two or more plasma discharge modules may be set as a set of module and provided by a plurality of modules.

The plasma power supply unit 41 serves to supply electric power to a plurality of plasma discharge modules 20a˜20d by the control signal of the control unit 42, and transmits high voltage power to generate plasma in an atmospheric pressure state.

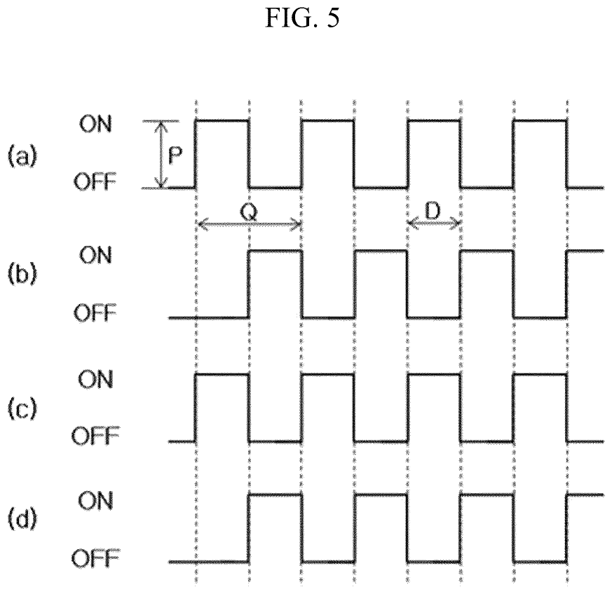

FIG. 5 is an exemplary diagram showing a method of applying electric power to the plasma discharge modules 20a˜20d shown in FIG. 3. For reference, FIGS. 5(a) to (d) show power supply cycle waveforms which show the state in which electric power is supplied to each of the four plasma discharge modules of FIG. 3.

Referring to FIG. 5, the control unit 42 is controlled so that the first plasma discharge module 20a and the second plasma discharge module 20b, and the third plasma discharge module 20c and the fourth plasma discharge module 20d form a pair and receive supply of electric power alternately at a fixed cycle. For example, as shown in FIG. 5, when the power supply of the first plasma discharge module 20a is turned ON, the other plasma discharge module 20b forming the pair is turned OFF with no power applied, and after a certain time has passed, when the electric power applied to the first plasma discharge module 20a is turned off, the electric power is supplied to the other second plasma discharge module 20b. In this manner, the electric power is similarly supplied alternately to the third and fourth plasma discharge modules 20c and 20d.

According to this configuration, each of the plasma discharge modules 20a to 20d receive supply of electric power alternately at a fixed cycle to provide continuity, so that plasma is generated continuously within the plasma reaction unit 21.

In addition, the time during which the plasma discharge modules 20a to 20d are periodically supplied with electric power and operated (ON) can be adjusted. Based on the concentration of ozone (O₃) contained in the inside air detected by the ozone sensor unit 22 shown in FIG. 2, the time is controlled within a duty ratio range of 20 to 50% based on one cycle as shown in FIG. 5(a), thereby capable of adjusting the generating amount of ozone (O₃) produced by the plasma.

In FIG. 5(a), P represents voltage, Q represents cycle, and D represents duty ratio.

According to the above configuration, when electric power is continuously supplied to a single or multiple plasma discharge modules at the same time, it is possible to prevent high heat generation and loss of discharge electrodes due to excessive energy concentration, which is advantageous in that it is possible to extend the performance and life of the plasma air purifying apparatus. In addition, the respective plasma discharge modules 20a to 20d can be evenly distributed and disposed within the plasma reaction unit 21, which is effective in achieving a more uniform purification effect on the sucked air to be purified.

Referring to FIG. 2, the catalytic filter unit 25 is communicated with the plasma reaction unit 21 and includes a catalytic agent for removing ozone (O₃) gas generated inside. When plasma is generated inside the plasma reaction unit 21, harmful gases to the human body, such as ozone (O₃) or nitrogen oxide (NOx), are generated together. At this time, the air containing the harmful gases generated is moved to the catalytic filter unit 25 by the blower fan 30, decomposed by reaction with the catalytic agent, and then discharged to the outside through the air discharge unit 34.

The catalytic agent used at this time includes 72 mol % or more of manganese dioxide (MnO₂) and 16 mol % or more copper oxide (CuO) as a main component, and may include other small amounts of potassium (K) and aluminum oxide (Al₂O₃). However, in order to increase the ozone (O₃) decomposition efficiency of the catalytic agent, it may be constituted in a pellet or ball shape having a diameter of 5 mm or less which contains 72 to 84 mol % of manganese dioxide (MnO₂) and 16 to 28 mol % of copper oxide (CuO), depending on environmental conditions such as temperature and flow rate.

In addition, the catalytic filter unit 25 is preferably a flat honeycomb filter type that incorporates a catalytic agent but allows easy gas passage.

On the other hand, as a method of rapidly promoting the ozone (O₃) decomposition reaction of the catalytic filter unit 25 and maximizing the decomposition rate, a method of applying titanium dioxide (TiO₂) photocatalytic particles to the surface of the catalytic agent and irradiating ultraviolet (UV) light thereto can be used. In this case, the titanium dioxide (TiO₂) photocatalyst is preferably a manganese dioxide (MnO₂) compound catalyst having a structure which is evenly applied or coated onto the surface of the catalyst at 50 to 80% in the form of small particles with a diameter of 100 nm or less, and the ultraviolet (UV) light irradiated thereto is preferably irradiated with ultraviolet (UV) light evenly over a wide area by using four or more semiconductor LEDs having a light source wavelength of 250 nm to 400 nm and an ultraviolet (UV) radiation output of 10 mW to 1,000 mW.

The ultraviolet (UV) light source used herein has generally been an ultraviolet (UV) lamp, but the ultraviolet (UV) lamp contains mercury (Hg), a substance harmful to the human body, which causes environmental pollution problems, and they have a short life of around 5,000 hours, which causes a problem that they need to be replaced frequently. However, in recent years, as semiconductor ultraviolet (UV) LEDs of various specifications have been developed and supplied, it is preferable to use ultraviolet (UV) LEDs that have a long life, good power efficiency, and a fast response time.

Generally, manganese dioxide (MnO₂)-copper oxide (CuO) compound catalytic agents have a disadvantage in that as the ambient temperature is lower, the ozone (O₃) decomposition efficiency decreases. As a method for complementing this, the disadvantage can be improved by increasing the activity of the catalytic reaction by applying titanium dioxide (TiO₂) photocatalyst to the catalytic agent and irradiating it with ultraviolet (UV) light. In addition, in this process, the photochemical reaction between ultraviolet (UV) light and the photocatalyst can be used to additionally once more remove microorganisms and harmful substances remaining in the air to be purified.

Referring to FIG. 2, the air sucked through the air suction unit 31 is purified by the chemical reaction of plasma in the plasma reaction unit 21, and the ozone (O₃) concentration generated during the purifying process and included in the inside air is detected by the ozone sensor unit 22, and based on this concentration value, the light output intensity and light quantity of the ultraviolet (UV) LED 23 incorporated into the UV irradiation unit 24 can be adjusted, which can reduce the power consumed by the UV irradiation unit 24, and has the effect of extending the life of the ultraviolet (UV) LED 23.

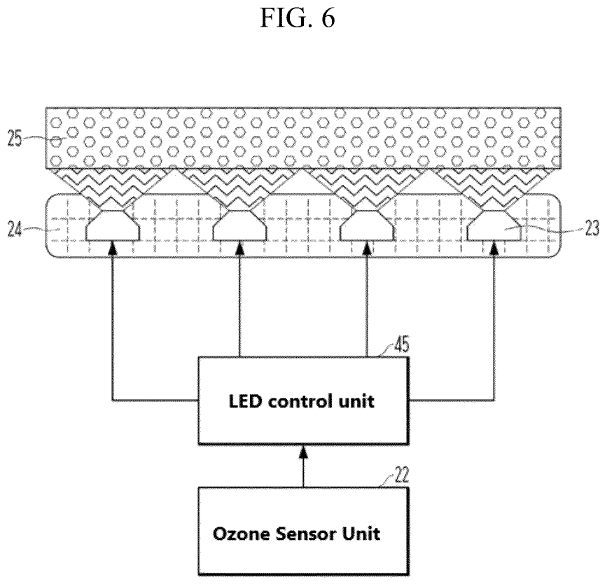

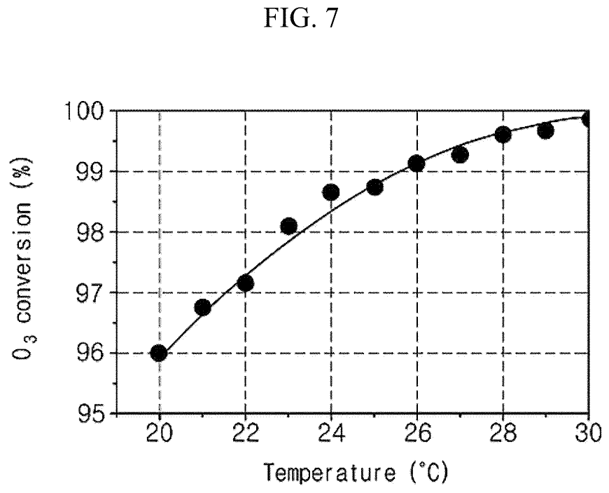

FIG. 7 is a graph showing the ozone (O₃) decomposition rate by the catalyst filter unit 25 and the ultraviolet (UV) irradiation unit 24 shown in FIG. 6.

As shown in FIG. 7, at a temperature of 20° C. or more, it showed a decomposition efficiency of 96% or more, and at a temperature of 30° C. or more, it showed a decomposition rate of nearly 100%. This shows that it is a significantly higher level under the same conditions, as compared to the commonly used high-temperature decomposition method, the method simply using manganese dioxide (MnO₂) as a catalytic agent, the method of decomposing using activated carbon as an adsorbent, or the like.

Accordingly, the plasma air purifying apparatus according to the preferred embodiment has the advantage of excellent decomposition efficiency of harmful gases such as ozone (O₃) and rapid purification treatment.

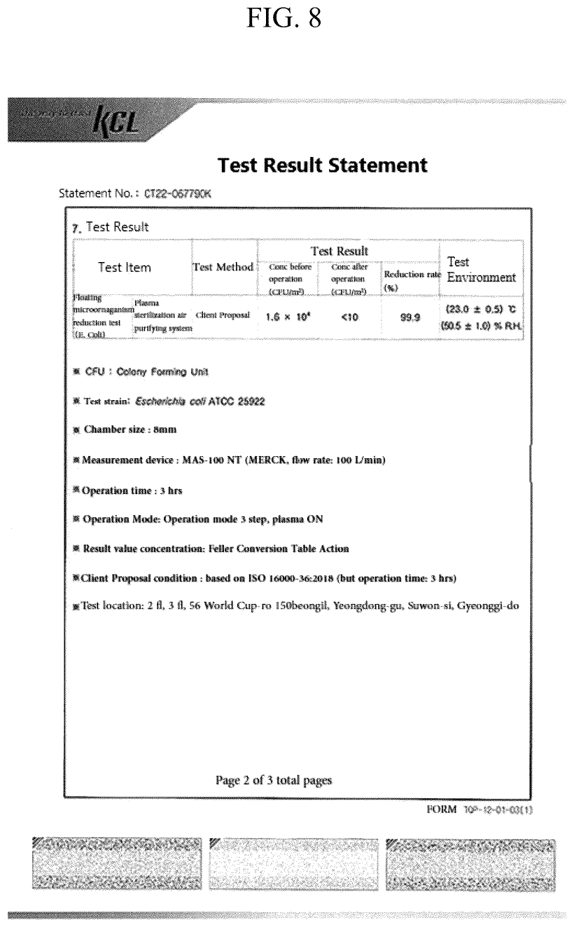

FIG. 8 shows the result of a sterilization test of microorganisms floating in indoor air by a plasma air purifying apparatus according to one embodiment.

Although the present invention has been described and illustrated with specific preferred embodiments above, it will be obvious that the present invention is not limited to the embodiments set forth above, and various modifications and imitations can be made to the embodiments by those skilled in the art without departing from the spirit and scope of the present invention.

1. An air purifying apparatus that uses atmospheric pressure plasma to kill microorganisms and remove harmful substances, the apparatus comprising:
an air purifying apparatus main body that sucks outside air, sterilizes floating microorganisms contained in the sucked air, removes harmful substances, etc., and then discharges them to the outside;
an air suction unit that sucks outside air into the inside of the air purifying apparatus main body;
an air discharge unit that discharges the sucked inside air to the outside;
at least two or more atmospheric pressure plasma discharge modules installed inside the air purifying apparatus main body;
a plasma reaction unit that performs the function of sterilizing microorganisms and removing harmful substances by a chemical reaction of the air sucked from the air suction part with the active species (including OH radical) and ozone (O₃) generated from the plasma discharge module;
a catalytic filter unit that removes residual ozone (O₃) generated in the plasma discharge module and remaining after the chemical reaction in the plasma reaction unit;
an ultraviolet (UV) irradiation unit that rapidly promotes the ozone (O₃) decomposition reaction of the catalytic filter unit;
an ozone sensor unit that detects the concentration of ozone (O₃) contained in inside air after a chemical reaction of the plasma reaction unit;
a power supply unit that supplies high voltage power to the plasma discharge module;
a control unit that controls the electric power supply of the electric power supply unit; and
a pre-filter and a HEPA filter installed between the air suction unit and the plasma reaction unit,
wherein air sucked in through the air suction unit is purified by passing through the pre-filter, HEPA filter, plasma reaction unit, ultraviolet irradiation unit, and catalytic filter unit in sequence, and then discharged to the air discharge unit,
wherein the catalytic agent of the above catalytic filter unit is a pellet or ball-shaped catalyst having a diameter of 5 mm or less containing 72 to 84 mol % of manganese dioxide (MnO₂) and 16 to 28 mol % of copper oxide (CuO), and includes a gas-passing catalytic filter in which a manganese dioxide (MnO₂)-copper oxide (CuO) compound catalytic agent is incorporated, the compound catalytic agent having a structure wherein titanium dioxide (TiO₂) photocatalyst particles with a diameter of 100 nm or less are evenly applied or coated at 50 to 80% onto the catalyst surface, and
wherein the irradiation intensity and light quantity of ultraviolet (UV) light that promotes the ozone (O₃) decomposition reaction in the catalytic filter unit are automatically adjusted based on the ozone (O₃) concentration contained in the inside air detected by the ozone sensor unit.

2. The air purifying apparatus according to claim 1, wherein the plasma discharge module receives supply of electric power at a fixed cycle and adjusts the time during which the apparatus operates (ON) within a duty ratio range of 20 to 50% based on one cycle, but is automatically adjusted based on the concentration of ozone (O₃) contained in the inside air detected by the ozone sensor unit.

3. The air purifying apparatus according to claim 1, wherein the ultraviolet (UV) generating device of the ultraviolet (UV) irradiation unit irradiates ultraviolet (UV) light to the catalytic filter unit by using at least four or more semiconductor LEDs having a light wavelength of 250 nm to 400 nm and an ultraviolet (UV) light emission output of 10 mW to 1,000 mW, so that ultraviolet (UV) light is irradiated onto the catalytic filter unit to exhibit the function of increasing the activity of the catalytic reaction and promoting the ozone (O₃) decomposition reaction.
